(19) United States
(12) Patent Application Publication
(10) Pub. No.: US 2016/0250043 A1
(43) Pub. Date: Sep. 1, 2016

(54) Device and method for perfusing porous biomaterials with biological liquids

(71) Applicant: BIOTECK S.P.A., Arcugnano (IT)

(72) Inventors: Mauro Fiorini, Granarolo dell'Emilia (BO) (IT); Fabrizio Raimondi, Castel d'Azzano (VR) (IT)

(73) Assignee: BIOTECK S.P.A., Arcugnano (VI) (IT)

(21) Appl. No.: 15/032,300

(22) PCT Filed: Sep. 11, 2014
(86) PCT No.: PCT/IB2014/064433

§ 371 Date: Apr. 26, 2016

(30) Foreign Application Priority Data
Nov. 19, 2013 (IT) VI2013A000277
Nov. 19, 2013 (IT) VI2013A000278

(51) Int. Cl. A61F 2/46; A01N 1/02

(52) U.S. Cl. CPC A61F 2/4644; A01N 1/0247; A61F 2002/4648; A61F 2002/2835

(57) ABSTRACT

A device for perfusing porous biomaterials with biological liquids includes a tubular body with an axial cavity defining a perfusion chamber for containing a porous biocompatible material, a connector at a distal end of the tubular body for receiving a perfusion liquid, a plunger having a distal end slidably movable in the cavity to withdraw air from the biomaterial, a vent for releasing the air withdrawn from the biomaterial and contained in the chamber, and a filter permitting selective passage of the air contained in the chamber toward the vent, while preventing leakage of the liquid to the outside. An air collector communicates with the chamber and the vent, and a control unit is associated with the plunger and designed to be actuated by the user to control the air flow from the air collector to the vent.

A method of perfusing porous biomaterials with biological liquids.

### FIELD OF THE INVENTION

The present invention generally finds application in the field of medical devices and particularly relates to a device for perfusing porous biomaterials with biological liquids.

The invention also relates to a method of perfusing porous biomaterials with biological liquids.

### BACKGROUND ART

Devices and methods are known to be used in the medical field of therapeutic treatments for perfusing a porous biomaterial with a perfusion liquid. The perfusion liquid may be selected from the group comprising aqueous solutions, bone marrow or derivatives thereof, antibiotic-containing solutions, peripheral blood, blood components or the like.

The use of perfused biological materials is particularly important in surgical treatment of muscoloskeletal tissue alterations and/or defects, for example caused by events of traumatic origin, neoplastic or degenerative diseases, delayed unions, infant deformities, or the like.

Particularly, these devices and methods promote perfusion of biomaterials designed to be grafted into the human body as substitutes for bones, cartilages, osteocartilages, tendons, ligaments or the meniscus and other connective tissues in general.

The devices and methods for perfusing biomaterials allow air to be withdrawn from the pores of the biomaterial and a given biological liquid to be filled therein to increase the contact surface and enhance liquid-biomaterial interaction.

The perfusion process must be carried out in an isolated environment, whereby devices have been developed for perfusing a biomaterial which are adapted to maintain the biomaterial and the fluid under high sterility conditions.

WO2009125282 discloses a device and a method for perfusing biomaterials which use a perfusion chamber for containing the material to be perfused, connected to a transfer chamber containing the biological liquid.

The perfusion chamber and the transfer chamber are sealingly coupled to each other and isolated from the outside environment. The transfer chamber essentially consists of a syringe having a liquid outlet in fluid communication with the perfusion chamber.

One drawback of this solution is the difficulty of releasing the air withdrawn from the biomaterial to the outside, due to full isolation of the perfusion and transfer chambers.

When the operator uses the device, he/she has to position the perfusion and transfer chambers in the vertical direction for the air that has been withdrawn from the biomaterial to expand into the end portion of the transfer chamber that has no liquid therein.

The presence of air in the transfer chamber as perfusion cycles are repeated further decreases the efficiency of the device and increases the risk that it may be reintroduced into the biomaterial to be perfused.

In view of obviating these drawbacks, devices and methods are known for perfusing biomaterial with biological liquid, which afford the air that has been withdrawn from the biomaterial during the perfusion cycle to be released to the outside.

US2008/0214998 discloses a device and method for perfusing biomaterial with a biological liquid which, in a particular embodiment, use a single perfusion chamber to contain both the material to be treated and the biological fluid.

Particularly, the device comprises a plunger held in the chamber, which is designed to be manually actuated by the user to decrease and/or increase pressure in the chamber, to assist the release of air from the porosities of the biomaterial and the introduction of liquid therein.

This device comprises a hydrophobic gasket associated with one end of the plunger and adapted to retain the liquid in the perfusion chamber and allow escape of air through the passages formed in the plunger.

A first drawback of this arrangement is that not a high fluid sealing action is provided between the hydrophobic gasket and the end of the plunger, which causes the risk of small liquid leakages during use of the device.

A further drawback of this arrangement is that the leakage of the perfusion liquid through the plunger often has to be compensated for by predetermined amounts of liquid.

This compensation may be particularly difficult because many therapeutic treatments require perfusion of the biomaterial with very small amounts of biological fluids that have been withdrawn from living patients by particularly complex and dangerous operations.

An additional drawback of this arrangement is that the compensation for such fluid leakage requires longer total perfusion times.

Another important drawback is that the fluid and the biomaterial are exposed to significant contamination due to the low fluid sealing action between the gasket and the plunger.

### DISCLOSURE OF THE INVENTION

The object of the present invention is to overcome the above drawbacks, by providing a device and a method for perfusing porous biomaterials with biological fluids, that is highly efficient and relatively cost-effective.

A particular object of the present invention is to provide a device and a method for perfusing porous biomaterials with biological liquids that afford optimized withdrawal of air from the biomaterial and saturation of the biomaterial with biological liquid.

Another object of the present invention is to provide a device and a method for perfusing porous biomaterials with biological liquids that afford high isolation of liquid from the outside environment, to considerably reduce leakages during use.

A further object of the present invention is to provide a device and a method for perfusing porous biomaterials with biological liquids that afford perfusion of the biomaterial with small amounts of liquid.

Another object of the present invention is to provide a device and a method for perfusing porous biomaterials with biological liquids that afford reduced perfusion times.

A further object of the present invention is to provide a device and a method for perfusing porous biomaterials with biological liquids that maintain the biomaterial and the liquid under aseptic conditions.

These and other objects, as clearly explained hereinafter, are fulfilled by a device for perfusing porous biomaterials with biological liquids as defined in claim 1, which comprises a tubular body with an axial cavity defining a perfusion chamber for containing a porous biocompatible material, a connector located at a distal end of the tubular body for introducing a perfusion liquid therein and a plunger with a distal end adapted to slide in the cavity to withdraw air from the biomaterial to be perfused.

Furthermore, the device comprises vent means for releasing the air withdrawn from the biomaterial and contained in the perfusion chamber to the outside, and filter means which permit selective passage of the air contained in the chamber toward the vent means, while preventing leakage of the liquid to the outside.

The device is characterized in that it comprises air collection means which communicate with the perfusion chamber and the vent means.

In order to control air flow from the collection means to the vent means control means are provided, which are associated with the plunger and are designed to be actuated by the user to control the air flow from the collection means to the vent means.

With this arrangement, the device affords optimized withdrawal of air from the biomaterial, while maintaining high isolation of the liquid from the outside environment and hence considerably reducing leakages thereof during use, and reducing the amount of the biological liquid required for perfusion.

In another aspect, the invention provides a method of perfusing porous biomaterials as defined in claim 11.

Advantageous embodiments of the invention are as defined in the dependent claims.

### BRIEF DESCRIPTION OF THE DRAWINGS

Further features and advantages of the invention will be more readily apparent upon reading of the detailed description of a preferred, non-exclusive embodiment of a device and a method for perfusing a porous biomaterial with perfusion liquids or the like, which are shown as a non-limiting example with the help of the annexed drawings, in which:

FIG. 1 is an exploded perspective view of a device of the invention;

FIG. 2 is an assembled perspective view of the device of FIG. 1;

FIG. 3 is a lateral view of the device of FIG. 1;

FIG. 4 is a broken away lateral view of the device of FIG. 1 in a first operating position;

FIG. 5 is an enlarged lateral view of a first portion of FIG. 4;

FIG. 6 is an enlarged lateral view of a second portion of FIG. 4;

FIG. 7 is a broken away lateral view of the device of FIG. 1 in a second operating position;

FIG. 8 is a broken away lateral view of the device of FIG. 1 in a third operating position;

FIG. 9 is a broken away front view of the device of FIG. 1;

FIG. 10 is a broken away and exploded lateral view of the device of FIG. 1;

FIG. 11 is a block diagram of the perfusion method of the invention.

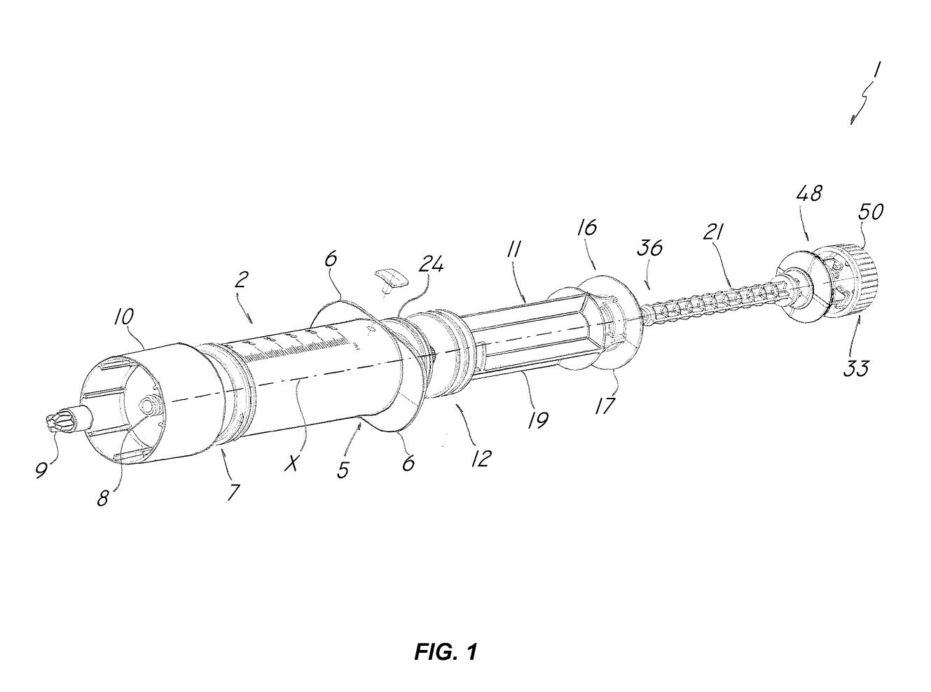

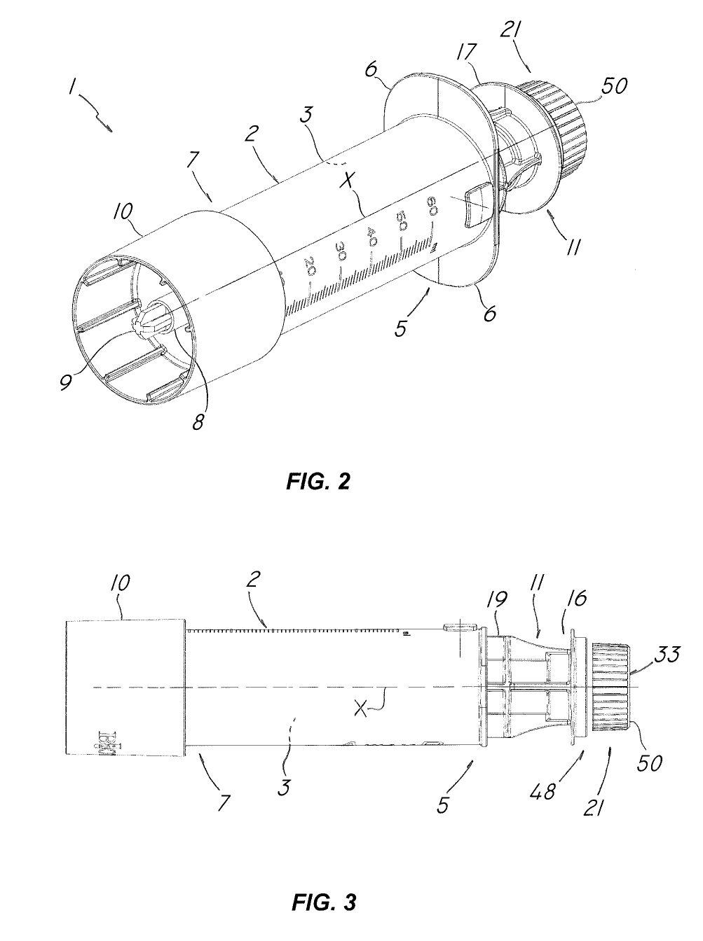

### DETAILED DESCRIPTION OF A PREFERRED EMBODIMENT

With reference to the above mentioned figures a device is shown, generally designated by numeral 1, for perfusing a porous biomaterial P with biological liquids L.

Particularly, the device 1 may be used for treating porous biomaterials P of a variety of types and shapes, which are designed to be grafted into the human body as substitutes for bones, cartilages, osteocartilages, tendons, ligaments or the meniscus or other connective tissues in general.

Perfusion liquids L which are typically used may be of biological origin, such as peripheral blood and derivatives thereof, bone marrow, bone marrow concentrate, adipose tissue-derived cell concentrates, physiological solutions containing antibiotics or other similar drugs.

In the embodiment of the figures, the device 1 is in syringe-like form and comprises a tubular body 2 with a longitudinal axis X and an axial cavity 3 defining a perfusion chamber 4 for holding a porous biomaterial P to be perfused.

The tubular body 2 has an open proximal end 5, having a pair of diametrically-opposed wings 6 for gripping by a user, and a distal end 7 with a connector 8 for introducing a perfusion liquid L into the perfusion chamber 4. The connector 8 may be closed off with a Luer lock cap 9 of per se known type.

Particularly, the connector 8 may be formed on a closing cover 10, which is removably mounted to the distal end 7 of the tubular body 2.

Advantageously, the removal of the closing cover 10 may provide access to the perfusion chamber 4 for introducing the porous biomaterial P therein before perfusion.

A plunger 11 is also provided, which may be equipped with longitudinal stiffening ribs for improved compressive and longitudinal tensile strength, and has a distal end 12 which is slidably movable in the cavity 3 for withdrawing the air contained in the biomaterial P to be perfused.

As shown in FIG. 5, an O-ring seal 14 is inserted in an annular groove 13 at the distal end of the plunger 12, for providing a sealing action on the surface 15 of the axial cavity 3 during its axial sliding motion.

In the embodiment of the figures, an annular flange 17 having a smaller size than the wings 6 is provided at the proximal end 16 of the plunger 11, for even easier gripping by a user.

The device further has vent means, generally referenced 18, for releasing the air withdrawn from the biomaterial P and collected in the perfusion chamber 4 to the outside.

The plunger 11 may comprise an elongate member 19 having a central axial through hole 20 for accommodating a stem 21 and an annular seat 22 fluidically connected to the hole 20 and located near the proximal end 16 of the plunger 11.

The fluid communication between the annular seat 22 and the axial hole 20 is ensured by the vent means 18 that shall permit air removal.

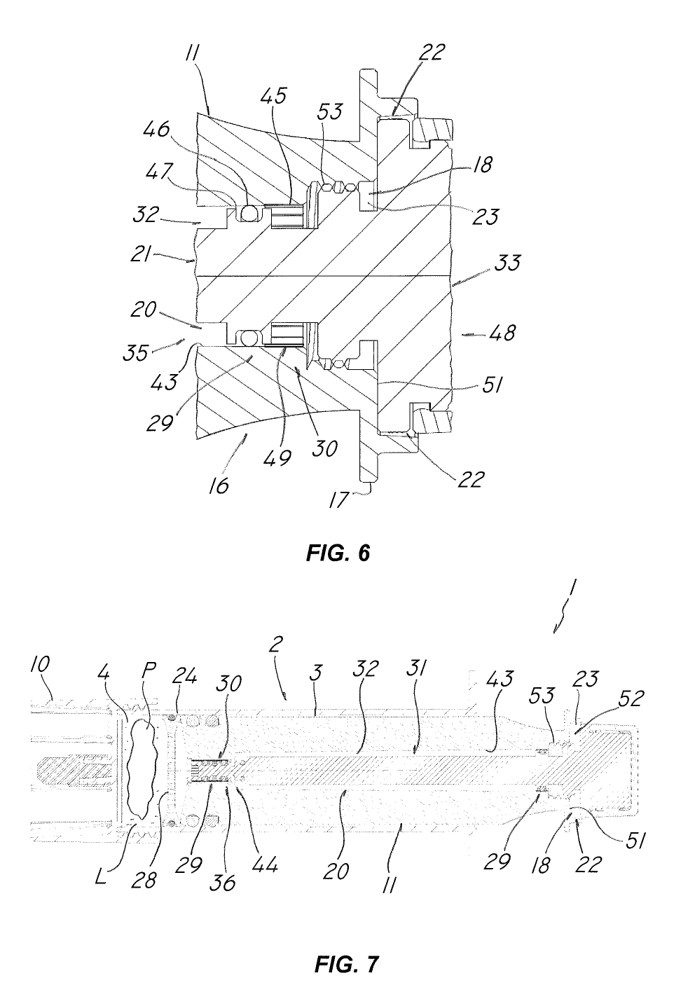

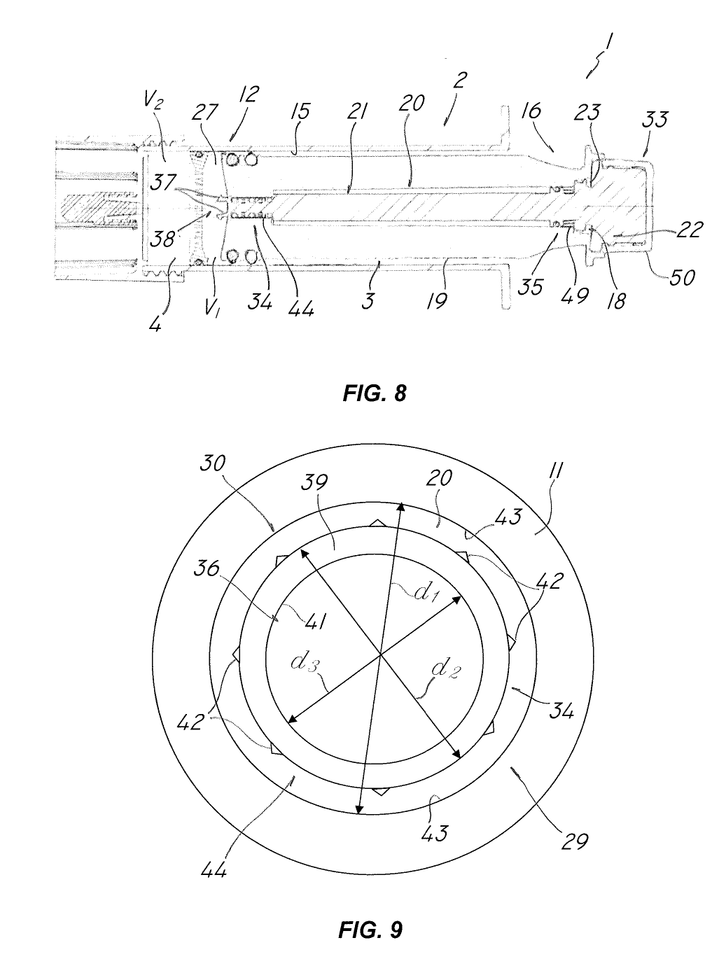

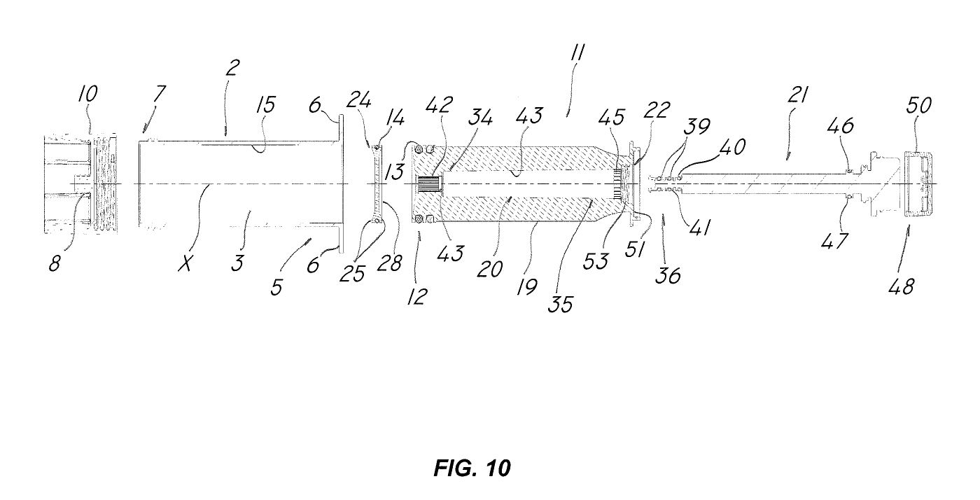

As best shown in FIG. 6, the vent means 18 may comprise an annular passageway 23, also formed at the distal end 16 of the plunger 11.

Conveniently, the perfusion chamber 4 may be defined by a substantially transverse partition 24 which is designed to be positioned in the cavity 3.

The partition 24 may substantially have a disk shape with a pair of slightly concave faces 25 facing the distal end 7 of the tubular body 2 and the plunger 11 respectively, and an annular peripheral seal 26, also of the O-ring type, for sealingly locking it in a predetermined axial position.

The distal end 12 of the plunger 11 may be slightly concave and spaced from the partition 24 to define a suction chamber 27 having a predetermined volume V₁ for containing the liquid L with the air that has been withdrawn from the biomaterial P.

Conveniently, the axial position of the partition 24 may be appropriately adjusted to change the volume V₂ of the perfusion chamber 4.

Thus, the volume V₂ of the chamber 4 may be adapted to the actual maximum size of the porous biomaterial P, thereby allowing minimization of the amount of liquid L required for perfusion thereof.

Preferably, the partition 24 may comprise at least one calibrated orifice 28 for promoting fluid communication of the perfusion chamber 4 with the suction chamber 27.

Filter means, generally referenced 29, are also provided, for permitting selective passage of the air contained in the perfusion chamber 4 toward the vent means 18, while preventing discharge or leakage of the perfusion liquid L to the outside.

Particularly, the filter means 29 may comprise at least one filter element 30 located downstream from the orifice 28.

The selected passage of air through the filter means 29 is facilitated by the longitudinal motion of the plunger 11 in the axial cavity 3.

When the plunger 11 is stopped, it may move away from the partition 24 thereby creating a negative pressure in the space vacated by the plunger 11.

Thus, a part of the liquid L contained in the perfusion chamber 4 will be sucked back into the suction chamber 27 through the orifice 28.

The later movement of the plunger 11 toward the partition 24 will pressurize the liquid L, thereby causing forced separation of the air removed from the pores of the biomaterial P through the filter means 29 that contact the liquid L.

The provision of the seal 14 along the walls of the plunger 11 will prevent liquid L from flowing back up along the walls thereof and will cause the flow of liquid L to be only conveyed toward the filter means 29.

A peculiar feature of the invention is that it comprises air collection means 31 which communicate with the perfusion chamber 4 and the vent means 18.

Particularly, the collection means 31 may consist of a substantially annular air space 32 in communication with the outside environment.

The air space 32 may be defined by a predetermined radial clearance g between the through hole 20 and the stem 21 upon insertion of the latter.

According to a further peculiar aspect of the invention, control means 33 are associated with the plunger 11 and are designed to be actuated by a user to control the air flow from the collection means 31 to the vent means 18.

Conveniently, as best shown in FIG. 9, the through hole 20 may have a substantially cylindrical shape, with a substantially uniform first inside diameter d₁, and may have a distal end portion 34, with a second inside diameter d₂, which is smaller than the first, and a proximal end portion 35.

The distal portion 34 may be adapted to accommodate a corresponding distal end portion 36 of the stem 21 having a third outside diameter d₃ which is slightly smaller than the second inside diameter d₂.

The distal end portion 36 accommodated in the distal portion 34 of the hole 20 may be provided with tapering lock projections 37 and a diametrical slit 38, for the projections 37 to be able to bend inwards and snap into the distal end portion 34 of the hole 20 of the plunger 11.

Advantageously, as best shown in FIG. 5, the filter means 29 may comprise a first annular seal 39 accommodated in a first annular seat 40 formed on the cylindrical wall 41 of the distal end portion 36 of the stem 21.

Furthermore, the filter means 29 may comprise a plurality of first longitudinal micro-channels 42 formed in angularly offset positions on the inner wall 43 of the distal end portion 34 of the through hole 20 of the plunger 11.

Thus, the first seal 39 may interact with the first micro-channels 42 to form a first labyrinth filter 44.

The radially offset arrangement of the micro-channels 42 may be better understood with the help of FIG. 9, although such micro-channels are not drawn to scale with respect to the dimensions of the other parts of the device.

Advantageously, the filter means 29 may comprise a plurality of second longitudinal micro-channels 45 formed in angularly offset positions on the inner wall 43 of the proximal portion 35 of the through hole 20, and at least one second seal 46 accommodated in a second annular seat 47 of the proximal end 48 of the stem 21.

Particularly, in a similar manner as the first seal 39, the second seal 46 may interact with the second micro-channels 45 to form a second labyrinth seal 49 which is longitudinally offset from the first filter.

Conveniently, both the first 42 and second 45 micro-channels may have micrometric dimensions, for example equal to or smaller than 100 μm, and are adapted to facilitate selective passage of air bubbles.

Preferably, the control means 33 may comprise an operating ring 50 located at the proximal end 48 of the stem 21 and adapted to be sealingly inserted in the annular seat 22 of the plunger 11.

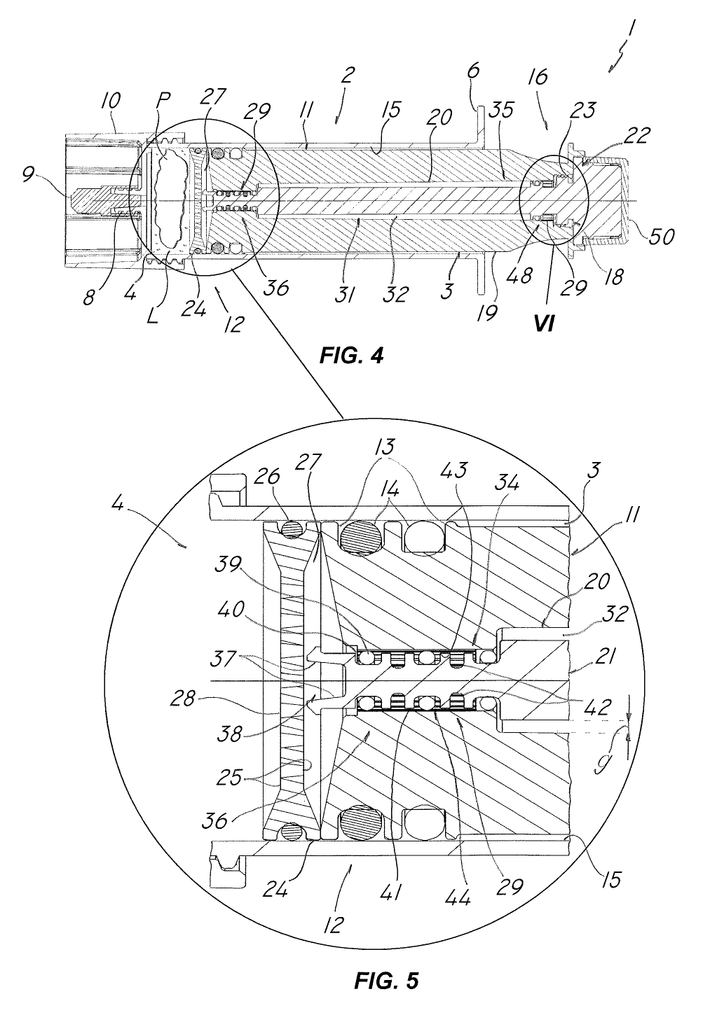

Particularly, the stem 21 is movable within the through hole 20 of the plunger 11 between a forward position, as shown in FIG. 4, in which the ring 50 contacts the bottom wall 51 of the annular seat 22 and blocks the through hole 20, and a backward position, as shown in FIG. 7, in which the ring 50 is separate from the seat 22 and opens the passageway 23 thereby defining a vent 52.

Conveniently, the stem 21 may have a locking member 53 near its proximal end 48, which is adapted to removably join the plunger 11 to the stem 21 when the latter is in the forward position.

The locking member 53 may allow the plunger 11 to reciprocate to create a negative pressure in the suction chamber 27 and convey air into the air space 32.

Furthermore, the locking member 53 may be releasable to move the stem 21 to the backward position and allow the air collected in the air space 32 to be released through the vent port 52.

Conveniently, the stem 21 may be moved between the forward position and the backward position a number of times to remove the air that has been previously withdrawn from the biomaterial P, while preventing it from being reintroduced into the pores thereof during the next perfusion cycle.

This arrangement increases the amount of liquid L that fills the pores of the biomaterial P thereby improving the overall efficiency of the perfusion process.

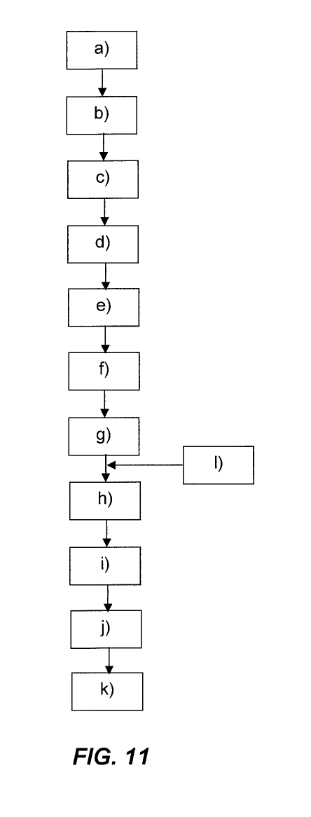

In a further aspect the invention relates to a method of perfusing biomaterials with biological liquids, as best shown in the block diagram of FIG. 11, which is adapted to be implemented by the device 1 for perusing a porous biomaterial P with biological liquids L.

The method basically comprises a step a) of providing the tubular body 2 with the inner cavity 3 defining the perfusion chamber 4.

Preferably, the closing cover 10 may be provided at the distal end 7 of the tubular body 2.

Once the cover 10 has been removed for access to the chamber 4 of the tubular body 2, a step b) will follow, in which a porous biomaterial P is placed in the chamber 4.

Possibly, the volume of the perfusion chamber 4 may be changed to fit the volume of the porous biomaterial P to be perfused by positioning the transverse partition 24, which is longitudinally translatable within the cavity 3.

This will optimize the volume of the chamber 4, and maximize the volume and amount of liquid L required for filling it.

Once the porous biomaterial P has been introduced into the perfusion chamber 4, the cover 10 may be mounted back to the distal end 7 of the tubular body 2 and the step c) of filling the chamber 4 with the perfusion liquid L may be carried out.

For this purpose, the connector 8 may be provided on the cover 10, for fluid communication with an external reservoir containing the biological liquid I, not shown, through a tube.

The chamber 4 containing the biomaterial P to be perfused may be filled by gravity. Alternatively, the perfusion chamber 4 may be filled using appropriate pumping means.

The step c) of filling the perfusion chamber 4 with the biological liquid L may be carried out under totally sterile conditions, for stable isolation of the liquid L from the outside environment.

Once the chamber 4 has been filled and the external tube has been removed, the connector 8 may be closed again using a Luer lock cap 9 of the per se known type, to prevent leakage of liquid L from the chamber 4 to the outside.

Then a step d) may be provided, in which the axially movable plunger 11 with the axial through hole 20 is introduced into the cavity 3 of the tubular body 2.

Conveniently, the plunger 11 is selected with an outside diameter that is slightly smaller than the inside diameter of the tubular body 2 and has O-rings 14 toward its distal end 12, such that it may sealingly slide in the inner cavity.

The method also includes a step e) in which the rigid stem 21 is accommodated in the axial hole 20.

The stem 21 is positioned in the axial hole 20 with a predetermined clearance, to thereby define the air collection space 32, in fluid communication toward its distal end 12 with the perfusion chamber 4, and toward the proximal end 16 of the plunger 11 with the outside.

Furthermore, the stem 21 in the axial hole 20 may have control means 33 for controlling fluid communication with the outside.

The control means 33 promote the release of withdrawn air to the outside during the perfusion process.

The method also comprises a step f) in which the stem 21 is positioned, relative to the plunger 11, in a forward locking position to prevent the passage of air from the air space 32 to the outside and facilitate mutual sliding thereof.

The stem 21 may be removably locked relative to the plunger 11 by separable locking members 53 which are located near the proximal end 16 of the plunger 11 and are designed to be accessed by a user.

Preferably, the locking step f) may be carried out by helical engagement of threads formed on the proximal end 36 of the inner wall 43 of the axial hole 20 of the plunger 11 with mating threads formed on the cylindrical wall of the proximal end 48 of the stem 21.

After the locking step f), the method comprises a step g) in which the stem-plunger assembly in locked relationship are reciprocated to repeatedly create suctions and compressions in the perfusion chamber 4.

The reciprocating motion of the assembly will pressurize the biological liquid L in the perfusion chamber 4 and cause it to effectively penetrate the pores of the biomaterial P.

Furthermore, once air has been withdrawn from the porous biomaterial P, the repeated reciprocating motion of the stem-plunger assembly will facilitate separation of air bubbles from the liquid L and conveyance thereof into the air collection space 32.

Advantageously, this arrangement will collect air in the air space 32 and prevent any communication of the inner portions of the tubular body 2 with the outside.

Preferably, the reciprocating motion of the assembly may be repeated as many times as is required for complete removal of air from the pores of the biomaterial P and the biological liquid L and for consequent saturation of the biomaterial P with the liquid.

Furthermore, as suction and compression steps are alternately repeated, the user will firmly tap his/her hand palm on the tubular body 2 to facilitate separation of the air bubbles that are still in the pores of the biomaterial P due to cavitation.

At the end of the perfusion process, the method includes a step h) in which the stem 21 is placed in a backward unlocking position relative to the plunger 11 to allow communication of the air space 32 with the outside and release air therein.

The stem 21 may be moved by providing and longitudinally displacing an operating ring 50 connected to the proximal end 48 of the stem 21. Advantageously, the ring 50 of the stem 21 may define the control means 33 for selectively promoting communication of the air collection space 32 with the outside.

Conveniently, communication between the air collection space 32 and the outside may take place thanks to the passageway 23 provided at the proximal end 16 of the plunger 11, where a seat 22 for the operating ring 50 may be also conveniently provided.

Advantageously, the passageway 23 may be closed by moving the stem 21 forward and entirely introducing the ring 50 into its seat 22.

Conversely, the passageway 23 may be opened by retracting the stem 21 and moving the ring 50 away from the seat 22 for the air contained in the air space to be released to the outside.

Controlled retraction of the stem 21 may be repeated by the user until the air contained in the air collection space 32 is entirely exhausted to the outside environment.

The method also includes a step i) in which the steps g) and h) are repeated as many times as is required for approximately complete removal of air from the pores of the biomaterial P and expulsion thereof.

After the repetition step i), there will be a step j) in which the liquid L is drained from the perfusion chamber 4 and a step k) in which the perfusion chamber 4 is opened and the completely treated porous biomaterial P is removed therefrom.

Preferably, as air enters and exits the air space 32, the method includes respective filtering steps I) for separating the air that has been withdrawn from the porous biomaterial P from the biological liquid L.

The filtering steps I) may be carried out using the longitudinal micro-channels 42, 45 that are formed at the end zones 34, 35 of the axial hole 20 and one or more annular seals 39, 46 mounted to the corresponding areas 36, 48 of the stem 21.

Particularly, these filtering steps I) may be carried out by interaction of the micro-channels 42, 45 with the annular seals 39, 46 such that micro-metric openings are created, to prevent leakage of liquids and allow selective passage of air.

The annular seals 39, 46 are adapted to directly interact with the inner wall 43 of the axial hole 20 to ensure a sealing action against leakage of biological liquid L toward the air space 32 and simultaneous formation of micro-metric openings for the air that has been withdrawn from the porous material P.

The device and the method for perfusing a porous biomaterial of the invention are susceptible of many changes and variants within the inventive principle disclosed in the annexed claims.

All the details and procedures thereof may be replaced by other technically equivalent parts and methods, and the materials may vary depending on different needs, without departure from the scope of the invention.

While the device and method of the invention have been described with particular reference to the annexed figures, the numerals referred to in the disclosure and claims are only used for the sake of a better intelligibility of the invention and shall not be intended to limit the claimed scope in any manner.

### INDUSTRIAL APPLICABILITY

The present invention finds industrial application in the field of medical devices and particularly for the provision of a device and method for perfusing porous biomaterials with biological liquids.

The invention claimed is:

1. A device for perfusing porous biomaterials with biological liquids, comprising:
a tubular body (2) with an axial cavity (3) defining a perfusion chamber (4) for containing a porous biomaterial (P);
a connector (8) located at a distal end (7) of said tubular body (2) for introducing a perfusion liquid (L) therein;
a plunger (11) having a distal end (12) slidably movable in said cavity (3) to withdraw air from the biomaterial (P) to be perfused;
a vent (18) for releasing the air withdrawn from the biomaterial (P) and contained in said chamber (4) to an outside;
a filter (29) for permitting selective passage of the air contained in said chamber (4) toward said vent (18), while preventing leakage of the liquid (L) to the outside; and
an air collector (31), which communicates with said chamber (4) and said vent (18), a control unit (33) being associated with said plunger (11) and designed to be actuated by a user to control air flow from said collector (31) to said vent (18).

2. The device as claimed in claim 1, wherein said plunger (11) comprises an elongate element (19) having an axial through hole (20) and adapted to accommodate a stem (21), and a seat (22) located near a proximal end (16) of said elongate element (19), said seat (22) and said through hole (20) being fluidically connected through said vent (18) comprising an annular passageway (23).

3. The device as claimed in claim 2, wherein said stem (21) has a predetermined radial clearance (g) relative to said axial through hole (20) to define a substantially annular air space (32) therewith, said collectors (31) consisting of said air space (32).

4. The device as claimed in claim 2, wherein said control unit (33) comprises an operating ring (50) located at a proximal end (48) of said stem (21) and adapted to be sealingly introduced into said seat (22) of said plunger (11).

5. The device as claimed in claim 5, wherein said stem (21) is movable within said through hole (20) of said plunger (11) between a forward position, in which said ring (50) contacts a bottom wall (51) of said seat (22) and blocks said through hole (20), and a backward position, in which said ring (50) is separate from said seat (22) and opens said passageway (23), thereby defining a second vent (52).

6. The device as claimed in claim 2, wherein said axial hole (20) has a substantially cylindrical shape, with a substantially uniform first inside diameter (d₁) and has a distal end portion (34), with a second inside diameter (d₂), which is smaller than the first inside diameter, and a proximal end portion (35), said distal portion (34) being adapted to accommodate a corresponding distal end portion (36) of said stem (21) having a third outside diameter (d₃) which is smaller than said second inside diameter (d₂).

7. The device as claimed in claim 4, wherein said chamber (4) is defined by a substantially transverse partition (24) which is designed to be adjustably positioned in said cavity (3) to adapt a volume (V₂) of said chamber (4) to a maximum size of the biomaterial (P) and minimize an amount of perfusion liquid (L), the distal end (12) of said plunger (11) being concave and spaced from said partition (24) to define a suction chamber (27) for sucking in liquid containing the air withdrawn from the biomaterial (P) to be perfused, said partition (24) having at least one calibrated orifice (28) which is adapted to allow communication between said perfusion chamber (4) and said suction chamber (27).

8. The device as claimed in claim 6, wherein said filter (29) comprises a plurality of first longitudinal micro-channels (42) formed in angularly offset positions on an inner wall (43) of said distal end portion (34) of said hole (20), and at least one first annular seal (39) accommodated in a first annular seat (40) of the distal end portion (36) of said stem (21) and adapted to interact with said first micro-channels (42) to form a first labyrinth filter (44).

9. The device as claimed in claim 8, wherein said filter (29) comprises a plurality of second longitudinal micro-channels (45) formed in angularly offset positions on an inner wall (43) of said proximal end portion (35) of said hole (20), and at least one second seal (46) accommodated in a second annular seat (47) of a proximal portion (48) of said stem (21) and adapted to interact with said second micro-channels (45) to form a second labyrinth filter (49), which is longitudinally offset from the first labyrinth filter.

10. The device as claimed in claim 4, wherein said stem (21) has a locking member (53) near said proximal end (48) for removably joining said plunger (11) to said stem (21), with said stem being in a forward position, and for allowing said plunger (11) to reciprocate to create a negative pressure in said suction chamber (27) and convey air into said air space (32), said locking member (53) being releasable to move said stem (21) to a backward position and allow the air collected in said air space (32) to be released through a second vent (52).

11. A method of perfusing porous biomaterials (P) with biological liquids (L), comprising the steps of:
a) providing a tubular body (2) with an inner cavity (3) defining a perfusion chamber (4);
b) positioning a porous biomaterial (P) in said chamber (4);
c) filling said chamber (4) with a perfusion liquid (L);
d) introducing an axially movable plunger (11), having an axial through hole (20), into said cavity (3);
e) accommodating a stem (21) in said hole (20), with a predetermined clearance to define an air collection space (32) communicating with said chamber (4) and with an outside and having a control unit (33) controlling communication with the outside;
f) positioning said stem (21), relative to said plunger (11), in a forward locking position to prevent passage of air from the air collection space (32) to the outside;
g) reciprocating the stem-plunger assembly in locked relationship to create repeated suctions and compressions in said chamber (4) and promote separation of air bubbles from the liquid and conveyance thereof toward said air collection space (32);
h) positioning said stem (21) relative to said plunger (11) in a backward unlocking position to permit communication of said air space (32) with the outside and release the air collected therein;
i) repeating steps g) and h) as many times as required for an approximately complete removal of air from pores of the porous biomaterial (P) and filling of the porous biomaterial with the perfusion liquid (L);
j) draining the liquid (L) from the perfusion chamber (4); and
k) opening the perfusion chamber (4) and removing the completely treated porous material (P).

12. The method as claimed in claim 11, wherein a plurality of longitudinal micro-channels (42, 45) are provided at an entry and exit of said air space (32), and are formed at end areas (34, 35) of said axial hole (20), and one or more annular seals (39, 49) are provided on corresponding areas (36, 48) of said stem (21), to form micro-metric openings, further comprising a filtering step l) in which the perfusion liquid (L) is filtered by said micro-metric openings to prevent leakage of liquids and allow selective passage of air.

13. The method as claimed in claim 11, wherein said stem (21) is locked relative to said plunger (11) by a separable locking member (53) which is located near a proximal end (16) of the plunger (11) and is designed to be accessed by a user.

14. The method as claimed in claim 11, wherein the backward and forward motions of said stem (21) in the axial hole (20) are obtained by providing an operating ring (50) connected to a proximal end (48) of said stem (21) and longitudinally moving the operating ring by a user.

15. The method as claimed in claim 14, wherein an annular passageway (23) is provided between said air space (32) and the outside at said proximal end (16) of said plunger (11) and a seat (22) is provided for said ring (50) at an exit of said passageway (23),
said passageway (23) being closed by moving said stem (21) forward and entirely introducing said ring (50) into said seat (22), the air collected in said air space (32) being released to the outside by moving said stem (21) backward and moving said ring (50) away from said seat (22), thereby opening said passageway (23).
